(19) United States
(12) Patent Application Publication
(10) Pub. No.: US 2024/0041936 A1
(43) Pub. Date: Feb. 8, 2024

(54) PREVENTION AND TREATMENT OF HAIR LOSS

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Niceville, FL (US)

(73) Assignee: CREATIVE MEDICAL TECHNOLOGIES, INC., Phoenix, AZ (US)

(21) Appl. No.: 18/366,516

(22) Filed: Aug. 7, 2023

Related U.S. Application Data
(60) Provisional application No. 63/395,836, filed on Aug. 7, 2022.

(51) Int. Cl. A61K 35/28; A61K 35/16; A61N 5/067; C07K 16/28; A61K 31/366; A61K 31/382; A61K 31/58; A61K 31/164

(52) U.S. Cl. CPC A61K 35/28; A61K 35/16; A61N 5/067; C07K 16/2887; A61K 31/366; A61K 31/382; A61K 31/58; A61K 31/164

(57) ABSTRACT

Disclosed are means, methods and compositions of matter for prevention of hair loss and/or regeneration of hair follicles utilizing stem cell-based approaches and synergies with stimulating and/or hormone modulating agents. In one embodiment the invention discloses specific platelet rich plasma (PRP) combinations alone or with bone marrow stem cells to augment hair restoration and stimulate expansion of hair follicle associated T regulatory cell.

### CROSS-REFERENCE TO RELATED APPLICATIONS

This application claims priority to U.S. Provisional Application No. 63/395,836, titled “Prevention and Treatment of Hair Loss” and filed Aug. 7, 2022, which is hereby incorporated by reference in its entirety.

### FIELD OF THE INVENTION

The invention is directed to methods of using regenerative cells to prevent and treat hair loss.

### BACKGROUND

It is known that the skin of an adult human is essentially covered with hair follicles and contains approximately five million hair follicles, with approximately 100,000-150,000 covering the scalp. The portions of human skin that lack visible hair contain, for the most part, hair follicles that produce “vellus hair” while certain other hair follicles may contain or produce no hair.

Only the glaborous skin on palmar and plantar aspects of hands and feet, respectively, and the lips and labia lack hair follicles. Only a minority of human hair follicles produce a hair fiber that can be readily appreciated visibly (a “terminal hair”) and these specialized follicles are localized on specific regions of skin; on the normal scalp, terminal hair follicles typically outnumber vellus hair follicles by 7:1. Accordingly, both the presence and absence of visible hair on human non-glaborous skin is mediated by regulation of activity of specialized follicles. Hair follicles, and particularly human hair follicles, are crypt structures comprised of distinct components, each comprised of several different specialized cell. In addition to the cells and structures associated with making and anchoring the hair shaft, the vast majority of hair follicles contain units called sebaceous glands (which produce sebum). Some hair follicles have apocrine glands attached to them, and are located in the axilla and other specific areas of the body. In addition to the hair shaft, the structures of the hair follicle include the follicular papilla (FP) and the germinative epithelium (GE) (together, the bulb). Scalp and certain other hair in humans tend to grow in follicular units. A follicular unit of scalp hair is typically composed of two to four terminal hair follicles; one, rarely two vellus hair follicles; their associated sebaceous glands, neurovascular plexus, an erector pilorum muscle and a circumferential band of adventitial collagen, termed the “perifolliculum”

### SUMMARY

Preferred embodiments are drawn to methods of preventing hair loss and/or stimulation of new hair growth comprising the steps of: a) identifying a patient in need of treatment; b) providing a bone marrow mononuclear cell/aspirate population into the area of affliction; c) administering platelet rich plasma into the area of affliction; and d) optionally providing adjuvant treatments.

Preferred methods include embodiments wherein administration of low-level laser irradiation is performed to augment production of local growth factors pre and/or post cell administration.

Preferred methods include embodiments wherein said laser irradiation of at least one wavelength, said wavelength(s) in a range between about 400 nanometers and about 1070 nanometers administered for a total energy of 100 .mu.W/cm to approximately 10 W/cm cm; and transplanting said irradiated cells to a patient in need.

Preferred methods include embodiments wherein said hair loss is caused by conditions selected from a group comprising of: a) Involutional alopecia; b) Androgenic alopecia; c) Alopecia areata; d) Alopecia universalis; e) Telogen effluvium; and f) Scarring alopecias.

Preferred methods include embodiments including administering minoxidil as a hair growth promoting agent.

Preferred methods include embodiments wherein, at 3 months after the integumental perturbation, the area of the scalp of the subject has at least 5%, 10%, 15%, 20%, 25%, 30%, 35%, 40%, 45%, 50%, 55%, 60%, 65%, 70%, 75%, 80%, 85%, 90%, 95%, or at least 100% more vellus hair compared to immediately before the integumental perturbation.

Preferred methods include embodiments further comprising: applying a wound healing dressing.

Preferred methods include embodiments wherein the wound healing dressing is non-occlusive.

Preferred methods include embodiments wherein the wound healing dressing is a cream, gel, lotion, emulsion, suspension, oil, non-aqueous solution, aqueous solution, or drop.

Preferred methods include embodiments wherein the wound healing dressing is applied for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 days after the integumental perturbation.

Preferred methods include embodiments wherein the minoxidil is administered once reepithelialization is completed, or 1, 2, 3, 4, 5, 6, 7, 8, 9, 10, 11, 12, 13, 14, 15, or 16 weeks after integumental perturbation.

Preferred methods include embodiments wherein the minoxidil is administered before and after integumental perturbation.

Preferred methods include embodiments wherein the minoxidil is administered for a period of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 weeks.

Preferred methods include embodiments wherein the minoxidil is administered following the new appearance of vellus hair on the area of the bald scalp that has been subjected to integumental perturbation.

Preferred methods include embodiments wherein said hair follicle adjuvant treatment is cromakalin.

Preferred methods include embodiments wherein said hair follicle adjuvant treatment is pinacidil.

Preferred methods include embodiments wherein said hair follicle adjuvant treatment is naminidil.

Preferred methods include embodiments wherein said hair follicle adjuvant treatment is diphenylcyclopropenone.

Preferred methods include embodiments wherein said hair follicle adjuvant treatment is tricomin.

Preferred methods include embodiments wherein said hair follicle adjuvant treatment is cyproterone acetate.

Preferred methods include embodiments wherein said hair follicle adjuvant treatment is danazol.

Preferred methods include embodiments wherein said hair follicle adjuvant treatment is flutamide.

Preferred methods include embodiments wherein said hair follicle adjuvant treatment is finasteride.

Preferred methods include embodiments wherein said hair follicle adjuvant treatment is turosteride.

Preferred methods include embodiments wherein said hair follicle adjuvant treatment is LY-191704.

Preferred methods include embodiments wherein said hair follicle adjuvant treatment is MK-306.

Preferred methods include embodiments wherein said hair follicle adjuvant treatment is dutasteride.

Preferred methods include embodiments wherein said hair follicle adjuvant treatment is a s-triazine.

Preferred methods include embodiments wherein said hair follicle adjuvant treatment is a pyridinopyran.

Preferred methods include embodiments wherein said hair follicle adjuvant treatment is a benzopyran.

Preferred methods include embodiments wherein said hair follicle adjuvant treatment is a thiane-1-oxide.

Preferred methods include embodiments wherein said hair loss is associated with a reduction in T regulatory cell number.

Preferred methods include embodiments wherein said T regulatory cells express FoxP3.

Preferred methods include embodiments wherein said T regulatory cells express membrane bound TGF-beta.

Preferred methods include embodiments wherein said T regulatory cells express membrane bound HLA-G.

Preferred methods include embodiments wherein said T regulatory cells express membrane bound IL-35.

Preferred methods include embodiments wherein said T regulatory cells express GITR.

Preferred methods include embodiments wherein said T regulatory cells express Fas ligand.

Preferred methods include embodiments wherein said T regulatory cells express membrane bound TRAIL.

Preferred methods include embodiments wherein said T regulatory cells secrete IL-10.

Preferred methods include embodiments wherein said platelet rich plasma is generated from peripheral blood.

Preferred methods include embodiments wherein said platelet rich plasma is generated from umbilical cord blood.

Preferred methods include embodiments wherein said platelet rich plasma is generated from mobilized peripheral blood.

Preferred methods include embodiments wherein said peripheral blood is mobilized by administration of G-CSF.

Preferred methods include embodiments wherein said peripheral blood is mobilized by administration of GM-CSF.

Preferred methods include embodiments wherein said peripheral blood is mobilized by administration of M-CSF.

Preferred methods include embodiments wherein said platelet rich plasma contains >10 pg/ml of interleukin 10.

Preferred methods include embodiments wherein said platelet rich plasma contains >30 pg/ml of FGF-1.

Preferred methods include embodiments wherein said platelet rich plasma contains >50 pg/ml of FGF-2.

Preferred methods include embodiments wherein an immune suppressive agent is administered prior to, concurrent with or subsequent to stem cell administration.

Preferred methods include embodiments wherein said immune suppressive agent is cyclophosphamide.

Preferred methods include embodiments wherein said immune suppressive agent is prednisone.

Preferred methods include embodiments wherein said immune suppressive agent is budesonide.

Preferred methods include embodiments wherein said immune suppressive agent is prednisolone.

Preferred methods include embodiments wherein said immune suppressive agent is tofacitinib.

Preferred methods include embodiments wherein said immune suppressive agent is cyclosporine.

Preferred methods include embodiments wherein said immune suppressive agent is tacrolimus.

Preferred methods include embodiments wherein said immune suppressive agent is everolimus.

Preferred methods include embodiments wherein said immune suppressive agent is azathioprine.

Preferred methods include embodiments wherein said immune suppressive agent is leflunomide.

Preferred methods include embodiments wherein said immune suppressive agent is Mycophenolate.

Preferred methods include embodiments wherein said immune suppressive agent is adalimumab.

Preferred methods include embodiments wherein said immune suppressive agent is anakinra.

Preferred methods include embodiments wherein said immune suppressive agent is certolizumab.

Preferred methods include embodiments wherein said immune suppressive agent is etanercept.

Preferred methods include embodiments wherein said immune suppressive agent is golimumab.

Preferred methods include embodiments wherein said immune suppressive agent is infliximab.

Preferred methods include embodiments wherein said immune suppressive agent is ixekizumab.

Preferred methods include embodiments wherein said immune suppressive agent is natalizumab.

Preferred methods include embodiments wherein said immune suppressive agent is rituximab.

Preferred methods include embodiments wherein said immune suppressive agent is secukinumab.

Preferred methods include embodiments wherein said immune suppressive agent is tocilizumab.

Preferred methods include embodiments wherein said immune suppressive agent is ustekinumab.

Preferred methods include embodiments wherein said immune suppressive agent is vedolizumab.

Preferred methods include embodiments wherein said immune suppressive agent is basiliximab.

Preferred methods include embodiments wherein said immune suppressive agent is daclizumab.

Preferred methods include embodiments to augment efficacy of hair transplantation.

Preferred methods include embodiments wherein the said platelet rich plasma has a hematocrit of less than 5%.

Preferred methods include embodiments wherein the said platelet rich plasma is leuko-rich.

Preferred methods include embodiments wherein the said platelet rich plasma is leuko-depleted.

Preferred methods include embodiments wherein the said platelet rich plasma is transformed into lyophilized platelet lysate.

Preferred methods include embodiments where the lyophilized platelet lysate is autologous.

Preferred methods include embodiments where the lyophilized platelet lysate is allogeneic.

Preferred methods include embodiments where the lyophilized platelet lysate is frozen at −4 to 20 degrees Celsius.

Preferred methods include embodiments where the lyophilized platelet lysate is room temperature greater than 16 degrees Celsius.

### DETAILED DESCRIPTION OF THE INVENTION

The invention teaches the utilization of regenerative cells alone and/or with therapeutic adjuvants to stimulate hair growth and prevent hair loss. Mature hair follicles (HF) progress through cycles of growth (anagen), degeneration (catagen), and then rest (telogen). Hair follicle stem cells (HFSCs), located at bulge of the resting HF, remain quiescent during telogen phase of the hair cycle. At the onset of anagen, some of HFSCs become proliferative and migrate downward to replenish the lower HF. The activation of HFSCs is tightly regulated by microenvironmental signals coming from their niche cells. The invention teaches the modulation of the microenvironment using exogenous regenerative cells and/or agents that stimulate activation of endogenous regenerative cells.

The term “alopecia” includes the involuntary complete or partial hair loss from the head or body of an individual and includes alopecia areata (AA), alopecia totalis (AT), alopecia universalis (AU), or chemotherapy-induced alopecia (CIA). Alopecia areata may include diffuse alopecia areata, alopecia areata monolocularis, alopecia areata multilocularis, and alopecia areata barbae. In some embodiments, alopecia does not include androgenetic alopecia (alopecia androgenetica, or male baldness) or post-chemotherapy alopecia (PCA).

Alopecia is the medical description of the loss of hair from the head or body, sometimes to the extent of baldness. Unlike the common aesthetic depilation of body hair, alopecia tends to be involuntary and unwelcome, e.g., androgenic alopecia. However, it may also be caused by a psychological compulsion to pull out one's own hair (trichotillomania) or the unforeseen consequences of voluntary hairstyling routines (mechanical “traction alopecia” from excessively tight ponytails or braids, or burns to the scalp from caustic hair relaxer solutions or hot hair irons). In some cases, alopecia is an indication of an underlying medical concern, such as iron deficiency. When hair loss occurs in only one section, it is known as “alopecia areata.” In human alopecia areata, hair is lost from some or all areas of the body, usually from the scalp. Because it causes bald spots on the scalp, especially in the first stages, it is sometimes called spot baldness. In 1%-2% of cases, the condition can spread to the entire scalp (alopecia totalis) or to the entire epidermis (alopecia universalis). Conditions resembling AA, and having a similar cause, occur also in other species. The most common type of alopecia areata involves hair loss in one or more round spots on the scalp. Hair may also be lost more diffusely over the whole scalp, in which case the condition is called diffuse alopecia areata. Alopecia areata monolocularis describes baldness in only one spot that may occur anywhere on the head. Alopecia areata multilocularis refers to multiple areas of hair loss. The disease may be limited only to the beard, in which case it is called alopecia areata barbae. If the individual loses all the hair on his/her scalp, the disease is then called alopecia areata totalis.

The term “Alopecia universalis” is when complete hair loss on the body occurs, similar to how hair loss associated with chemotherapy sometimes affects the entire body.

The term “Androgenic alopecia” (also known as androgenetic alopecia or alopecia androgenetica) is a common form of hair loss in both female and male humans, chimpanzees, and orangutans. In male humans in particular, this condition is also commonly known as male pattern baldness. Hair is lost in a well-defined pattern, beginning above both temples. Over time, the hairline recedes to form a characteristic “M” shape. Hair also thins at the crown of the head. Often a rim of hair around the sides and rear of the head is left, or the condition may progress to complete baldness. The pattern of hair loss in women differs from male pattern baldness. In women, the hair becomes thinner all over the head, and the hairline does not recede. Androgenic alopecia in women rarely leads to total baldness.

The term “preventing alopecia” includes the arresting of or suppression of hair loss associated with alopecia prior to its occurrence.

The language “mitigating alopecia” or “treating alopecia” includes reducing the severity of the hair loss associated with alopecia or reducing the extent of the hair loss associated with of alopecia. In some embodiments, mitigating or treating alopecia includes the amelioration of alopecia.

The term “administering” includes providing one or more doses of the regenerative cells to individual in an amount effective to prevent or treat alopecia. Optimal administration rates for a given protocol of administration of the regenerative cells can ascertained by those skilled in the art using conventional dosage determination tests conducted with regard to the specific compounds being utilized, the particular compositions formulated, the mode of application, the particular site of administration and the like.

The language “topically administering” includes delivering one or more doses of the vitamin D compound to the skin of the individual in an amount effective to treat or prevent alopecia.

The term “skin” refers to a structure containing many specialized cells and structures, and has various important functions, such as serving as a protective barrier that interfaces with the environment, helping to maintain the proper body temperature, gathering sensory information from the environment, and playing an active role in the immune system. The skin has three layers—the epidermis, dermis, and subcutaneous tissue. The epidermis is the outer layer of skin. Its thickness varies in different types of skin. It is the thinnest on the eyelids at about 0.05 mm and the thickest on the palms and soles at about 1.5 mm. From bottom to top, the epidermis contains five layers: stratum basale, stratum spinosum, stratum granulosum, stratum licidum (optional in some skins), and stratum corneum.

The term stratum basale is the bottom layer of keratinocytes in the epidermis and is responsible for constantly renewing epidermal cells. This layer contains just one row of undifferentiated columnar stem cells that divide very frequently. Half of the cells differentiate and move to the next layer to begin the maturation process. The other half stay in the basal layer and divide repeatedly to replenish the basal layer. Cells that move into the spinosum layer (also called prickle cell layer) change from being columnar to polygonal. In this layer, the cells start to synthesize keratin. The cells in the stratum granulosum, or granular layer, have lost their nuclei and are characterized by dark clumps of cytoplasmic material. There is a lot of activity in this layer as keratin proteins and water-proofing lipids are being produced and organized. The stratum lucidum layer is only present in thick skin where it helps reduce friction and shear forces between the stratum corneum and stratum granulosum. The cells in the stratum corneum layer are known as corneocytes. These cells have flattened out and are composed mainly of keratin protein which provides strength to the layer but also allows the absorption of water. The structure of the stratum corneum layer looks simple, but this layer is responsible for maintaining the integrity and hydration of the skin—a very important function.

The term dermis also varies in thickness depending on the location of the skin. It is about 0.3 mm on the eyelid and about 3.0 mm on the back. The dermis is composed of three types of tissue that are present throughout—not in layers: collagen, elastic tissue, and reticular fibers. The two layers of the dermis are the papillary and reticular layers. The upper, papillary layer, contains a thin arrangement of collagen fibers. The lower, reticular layer, is thicker and made of thick collagen fibers that are arranged parallel to the surface of the skin. The dermis contains many specialized cells and structures. For example, blood vessels and nerves course through this layer. The hair follicles are also situated in this layer with the erector pili muscle that attaches to each follicle. A portion of the hair follicle also contains stem cells capable of regrowing damaged epidermis. Stem cells may be present at the dermal-epidermal junction (DEJ). Sebaceous (oil) glands and apocrine (scent) glands are associated with the follicle. This layer also contains eccrine (sweat) glands, but they are not associated with hair follicles. The subcutaneous tissue is a layer of fat and connective tissue that houses larger blood vessels and nerves. This layer is important in the regulation of temperature of the skin itself and the body. The size of this layer varies throughout the body and from person to person.

Accordingly, as used herein, “epidermis” includes all five of its layers (when present), including the junction layer between epidermis and dermis (e.g., dermal-epidermal junction or DEJ), and stem cells that regenerates the epidermal layers (e.g., follicular stem cells and epidermal stem cells).

In one embodiment bone marrow cells are collected in the form of bone marrow mononuclear cells/aspirate and administered by a microneedling process into the scalp of a patient suffering from alopecia. Prior to administration of said cells adjuvants are added either systemically and/or locally to enhance therapeutic efficacy. In some embodiments adjuvants include administration of NK-kappa B inhibitors and/or antioxidants. Example of useful agents include Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+istonic).

In one embodiment prior to stem cell administration, 0.1 to about 10% of the anti-alopecia agent is administered, from about 0.1 to 10% of the at least one dermal penetration enhancer and from about 45 to 99.8% of a volatile liquid. In another preferred form the volatile liquid is ethanol, isopropanol or mixture thereof in the range of about 80 to 98%. Administration is performed at minimum 4 hours prior to cell therapy in order for the enhancing agent not to interfere with stem cell viability. In yet another form of the invention the drug delivery system comprises, on a weight basis, from about 1 to 5% of an anti-alopecia agent, from about 1 to 5% of the dermal penetration enhancer, from about 45 to 90% ethanol, isopropanol or mixture thereof, 5 to 45% water; and optionally 0.5 to 5% of a thickening agent. In some variants of the invention, the penetration enhancer may be applied before or after the application of the anti-alopecia agent, if desired. The present invention also provides a method for administering at least one systemic or locally acting anti-alopecia agent to a human which comprises applying an effective amount of the anti-alopecia agent in the form of the drug delivery system of the present invention. In one embodiment of the invention regenerative cells such as bone marrow mononuclear cells are administer with anti-alopecia agents which include, but are not limited to, minoxidil, cromakalin, pinacidil, naminidil, diphenylcyclopropenone, tricomin, antiandrogen agents such as cyproterone acetate, danazol and flutamide, 5-alpha reductase inhibitors such as finasteride, turosteride, LY-191704, MK-306 and dutasteride (U.S. Pat. No. 4,377,584), and those compounds selected from the classes of s-triazines, benzopyrans, pyridinopyrans and thiane-1-oxides or pharmaceutically acceptable salts or derivatives of any one of the aforementioned.

Other agents useful for enhancement of the anti-alopecia properties of bone marrow include cytokines that increase T regulatory cell numbers. These include low dose IL-2, IL-6, IL-27, Myosin 1, IL-33, Hypoxia Inducible Factor-1, Guanylate Binding Protein Isoform I, Aminolevulinate delta synthase 2, AMP deaminase, IL-17, DNAJ-like 2 protein, Cathepsin L, Transcription factor-20, M31724, pyenylalkylamine binding protein; HEC, GA17, arylsulfatase D gene, arylaulfatase E gene, cyclin protein gene, pro-platelet basic protein gene, PDGFRA, human STS WI-12000, mannosidase, beta A, lysosomal MANBA gene, UBE2D3 gene, Human DNA for Ig gamma heavy-chain, STRL22, BHMT, Homo sapiens Down syndrome critical region, FI5613 containing ZNF gene family member, IL8, ELFR, Homo sapiens mRNA for dual specificity phosphatase MKP-5, Homo sapiens regulator of G protein signaling 10 mRNA complete, Homo sapiens Wnt-13 Mma, Homo sapiens N-terminal acetyltransferase complex ard1 subunit, ribosomal protein L15 mRNA, PCNA mRNA, ATRM gene exon 21, HR gene for hairless protein exon 2, N-terminal acetyltransferase complex and 1 subunit, HSM801431 homo sapiens mRNA, CDNA DKFZp434N2072,RPL26, and HR gene for hairless protein, regulator of G protein signaling.

In some embodiments platelet rich plasma is utilized as an anti-alopecia agent that is administered prior to and/or subsequent to bone marrow cell/aspirate administration. In drug delivery systems according to the present invention a pharmaceutical compounding agent, co-solvent, surfactant, emulsifier, antioxidant, preservative, stabilizer, diluent or a mixture of two or more of said components may be incorporated in these systems as is appropriate to the particular route of administration and dosage form. Pharmaceutical compound is administered in the form of platelet rich plasma. The amount and type of components used should be compatible with the dermal penetration enhancers of this invention as well as with the anti-alopecia agent. A co-solvent or other standard adjuvant, such as a surfactant, may be required to maintain the anti-alopecia agent in solution or suspension at the desired concentration. The pharmaceutical compounding agents can include paraffin oils, esters such as isopropyl myristate, ethanol, silicone oils and vegetable oils. These are preferably used in the range 1 to 50%. Surfactants such as ethoxylated fatty alcohols, glycerol mono stearate, phosphate esters, and other commonly used emulsifiers and surfactants preferably in the range of 0.1 to 10% may be used, as may be preservatives such as hydroxybenzoate esters for preservation of the compound preferably in amounts of 0.01% to 0.5%. Typical co-solvents and adjuvants may be ethyl alcohol, isopropyl alcohol, acetone, dimethyl ether and glycol ethers such as diethylene glycol mono ethyl ether. These may be used in amounts of 1 to 50%. Because of the effect of the penetration enhancer of the invention, the dosage of the anti-alopecia agent may often be less than that conventionally used. It is proposed that, a dosage near the lower end of the useful range of the particular anti-alopecia agent may be employed initially and increased as indicated from the observed response if necessary. The concentration of anti-alopecia agent used in the drug delivery system will depend on its properties and may be equivalent to that normally utilised for the particular anti-alopecia agent in conventional formulations. Both the amount anti-alopecia agent and the amount of penetration enhancer will be influenced by the type of effect desired. Where it is desired to achieve higher local concentration of an anti-alopecia agent such as platelet rich plasma , proportionately higher concentrations of the enhancer of the invention may be required in the topical drug delivery system of the present invention, and the amount of anti-alopecia agent included in the composition should be sufficient to provide the tissue level desired. The concentration of absorption/penetration enhancer may be in the range from 10-10,000 weight percent of absorption/penetration enhancer based upon the weight of anti-alopecia agent. The ratio of penetration enhancer to anti-alopecia agent may vary considerably and will be governed as much as anything, by the pharmacological results that are required to be achieved. In principle, it is desirable that as little absorption enhancer as possible is used. On the other hand, for some anti-alopecia agents, it may well be that the upper range of 10,000% by weight will be required. It is preferred that the penetration enhancer and anti-alopecia agent are in approximately equal proportions. A particular advantage of the drug delivery system of the present invention is that patient compliance is improved as the system does not occlude the skin. As a result local irritation and allergic sensitization problems arising from prolonged exposure of the skin to both the delivery system of occlusive transdermal patch devices and the adhesive used to affix these patches to the skin are reduced.

In the preparation of platelet rich plasma for administration various excipients may by utilized. Examples include 1,2,6-hexanetriol, alkyltriols, alkyldiols, acetyl monoglycerides, tocopherol, alkyl dioxolanes, p-propenylanisole, anise oil, apricot oil, dimethyl isosorbide, alkyl glucoside, benzyl alcohol, bees wax, benzyl benzoate, butylene glycol, caprylic/capric triglyceride, caramel, cassia oil, castor oil, cinnamaldehyde, cinnamon oil, clove oil, coconut oil, cocoa butter, cocoglycerides, coriander oil, corn oil, coriander oil, corn syrup, cottonseed oil, cresol, cyclomethicone, diacetin, diacetylated monoglycerides, diethanolamine, dietthylene glycol monoethyl ether, diglycerides, ethylene glycol, eucalyptus oil, fat, fatty alcohols, flavors, liquid sugars ginger extract, glycerin, high fructose corn syrup, hydrogenated castor oil, IP palmitate, lemon oil, lime oil, limonene, milk, monoacetin, monoglycerides, nutmeg oil, octyldodecanol, olive alcohol, orange oil, palm oil, peanut oil, PEG vegetable oil, peppermint oil, petrolatum, phenol, pine needle oil, polypropylene glycol, sesame oil, spearmint oil, soybean oil, vegetable oil, vegetable shortening, vinyl acetate, wax, 2-(2-(octadecyloxy)ethoxy)ethanol, benzyl benzoate, butylated hydroxyanisole, candelilla wax, carnauba wax, ceteareth-20, cetyl alcohol, polyglyceryl, dipolyhydroxy stearate, PEG-7 hydrogenated castor oil, diethyl phthalate, diethyl sebacate, dimethicone, dimethyl phthalate, PEG fatty acid esters, PEG-stearate, PEG-oleate, PEG laurate, PEG fatty acid diesters, PEG-dioleate, PEG-distearate, PEG-castor oil, glyceryl behenate, PEG glycerol fatty acid esters, PEG glyceryl laurate, PEG glyceryl stearate, PEG glyceryl oleate, hexylene glycerol, lanolin, lauric diethanolamide, lauryl lactate, lauryl sulfate, medronic acid, methacrylic acid, multisterol extract, myristyl alcohol, neutral oil, PEG-octyl phenyl ether, PEG-alkyl ethers, PEG-cetyl ether, PEG-stearyl ether, PEG-sorbitan fatty acid esters, PEG-sorbitan diisosterate, PEG-sorbitan monostearate, propylene glycol fatty acid esters, propylene glycol stearate, propylene glycol, caprylate/caprate, sodium pyrrolidone carboxylate, sorbitol, squalene, stear-o-wet, triglycerides, alkyl aryl polyether alcohols, polyoxyethylene derivatives of sorbitan-ethers, saturated polyglycolyzed C8-C10 glycerides, N-methyl pyrrolidone, honey, polyoxyethylated glycerides, dimethyl sulfoxide, azone and related compounds, dimethylformamide, N-methyl formamaide, fatty acid esters, fatty alcohol ethers, alkyl-amides (N,N-dimethylalkylamides), N-methyl pyrrolidone related compounds, ethyl oleate, polyglycerized fatty acids, glycerol monooleate, glyceryl monomyristate, glycerol esters of fatty acids, silk amino acids, PPG-3 benzyl ether myristate, Di-PPG2 myreth 10-adipate, honeyquat, sodium pyroglutamic acid, abyssinica oil, dimethicone, macadamia nut oil, limnanthes alba seed oil, cetearyl alcohol, PEG-50 shea butter, shea butter, aloe vera juice, phenyl trimethicone, hydrolyzed wheat protein, and combinations thereof. Other agents useful for administration of said platelet rich plasma include solidifying agent such as polyvinyl alcohol, esters of polyvinylmethylether/maleic anhydride copolymer, neutral copolymers of butyl methacrylate and methyl methacrylate, dimethylaminoethyl methacrylate-butyl methacrylate-methyl methacrylate copolymers, ethyl acrylate-methyl methacrylate-trimethylam monioethyl methacrylate chloride copolymers, prolamine (Zein), pregelatinized starch, ethyl cellulose, fish gelatin, gelatin, acrylates/octylacrylamide copolymers, and combinations thereof. Other solidifying agent includes at least one member selected from the group consisting of ethyl cellulose, hydroxy ethyl cellulose, hydroxy methyl cellulose, hydroxy propyl cellulose, hydroxypropyl methyl cellulose, carboxymethyl cellulose, methyl cellulose, polyether amides, corn starch, pregelatinized corn starch, polyether amides, shellac, polyvinyl pyrrolidone, polyisobutylene rubber, polyvinyl acetate phthalate and combinations thereof. Furthermore, other solidifying agent include ammonia methacrylate, carrageenan, cellulose acetate phthalate aqueous, carboxy polymethylene, cellulose acetate (microcrystalline), cellulose polymers, divinyl benzene styrene, ethylene vinyl acetate, silicone, guar gum, guar rosin, gluten, casein, calcium caseinate, ammonium caseinate, sodium caseinate, potassium caseinate, methyl acrylate, microcrystalline wax, polyvinyl acetate, PVP ethyl cellulose, acrylate, PEG/PVP, xantham gum, trimethyl siloxysilicate, maleic acid/anhydride colymers, polacrilin, poloxamer, polyethylene oxide, poly glactic acid/poly-I-lactic acid, turpene resin, locust bean gum, acrylic copolymers, polyurethane dispersions, dextrin, polyvinyl alcohol-polyethylene glycol co-polymers, methyacrylic acid-ethyl acrylate copolymers, methacrylic acid and methacrylate based polymers such as poly(methacrylic acid), and combinations thereof.

Adjuvant therapies to enhance efficacy of discussed formulations include agents known in the art to prevent hair loss. These include, but are not limited to finasteride, dutasteride (e.g., Avodart), turosteride, bexlosteride, izonsteride, epristeride, epigallocatechin, MK-386, azelaic acid, FCE 28260, and SKF 105,111, ketoconazole, fluconazole, spironolactone, flutamide, diazoxide, 17-alpha-hydroxyprogesterone, 11-alpha-hydroxyprogesterone, ketoconazole, RU58841, dutasteride (marketed as Avodart), fluridil, or QLT-7704, an antiandrogen oligonucleotide, a prostaglandin F2.alpha. analogs, prostaglandin analogs, a prostaglandin, bimatoprost (e.g., Latisse, Lumigan), latanoprost (trade name Xalatan), travoprost (trade name Travatan), tafluprost, unoprostone, dinoprost (trade name Prostin F2 Alpha), AS604872, BOL303259X, PF3187207, carboprost (trade name Hemabate), kopexil (for example, the product Keranique.™.), CaCl2, botilinum toxin A, adenosine, ketoconazole, DoxoRx, Docetaxel, FK506, GP11046, GP11511, LGD 1331, ICX-TRC, MTS-01, NEOSH101, HYG-102440, HYG-410, HYG-420, HYG-430, HYG-440, spironolactone, CB-03-01, RK-023, Abatacept, Viviscal.®., MorrF, ASC-J9, NP-619, AS101, Metron-F-1, PSK 3841, Targretin (e.g. 1% gel), MedinGel, PF3187207, BOL303259X, AS604872. THG11331, PF-277343, PF-3004459, Raptiva, caffeine, coffee, a herb (such as, e.g., saw palmetto, Glycine soja, Panax ginseng, Castanea sativa, Arnica montana, Hedera helix Geranium maculatum), triamcinolone acetonide, a topical irritant (e.g., anthralin) or sensitizer (e.g., squaric acid dibutyl ester [SADBE] or diphenyl cyclopropenone [DPCP]), clomipramine, unsaturated fatty acids (e.g. gamma linolenic acid), a fatty acid derivative, a thickener (such as, e.g. carbomer, glycol distearate, cetearyl alcohol), a hair loss concealer, niacin, nicotinate esters and salts, adenosine, methionine, an androgen receptor inhibitor, a copper peptide, a compound with superoxide dismutation activity, an agent that increases nitric oxide production (e.g. arginine, citrulline, nitroglycerin, amyl nitrite, or sildenafil (Viagra)), a compound that mobilizes bone marrow-derived stem cells (e.g., growth factors such as G-CSF and/or chemical agents such as plerixafor (Mozobil.®.)), a compound that regulates the differentiation of stem cells into gender-specific specialized human hair follicles (e.g., finasteride, fluconazole, spironolactone, flutamide, diazoxide, 11-alpha-hydroxyprogesterone, ketoconazole, RU58841, dutasteride, fluridil, or QLT-7704, an antiandrogen oligonucleotide, cyoctol, topical progesterone, topical estrogen, cyproterone acetate, ru58841, combination 5.alpha.-reductase inhibitors, oral contraceptive pills), an antiestrogen, an estrogen, or estrogen-like drug, an anti-oxidants (e.g., glutathione, ascorbic acid, tocopherol, uric acid, or polyphenol antioxidants), inhibitors of reactive oxygen species (ROS) generation (e.g., superoxide dismutase inhibitors; stimulators of ROS breakdown, such as selenium; mTOR inhibitors, such as rapamycin: or sirtuins or activators thereof, such as resveratrol, or other SIRT1. SIRT3 activators, or nicotinamide inhibitors), an agent that induces an immune response or causes inflammation (e.g., tetanus toxoid, topical non-specific irritants (anthralin), or sensitizers (squaric acid dibutyl ester [SADBE] and diphenyl cyclopropenone [DPC1]), and an antiapoptotic compound.

In some embodiments of the invention, growth factors are added to enhance viability of the hair follicle unit, and/or promote mitosis. Said growth factors are selected from a group comprising of: BLC, Eotaxin-1, Eotaxin-2, G-CSF, GM-CSF, 1-309, ICAM-1, IFN-gamma, IL-1 alpha, IL-1 beta, IL-1 ra, IL-2, IL-4, IL-5, IL-6, IL-6 sR, IL-7, IL-8, IL-10, IL-11, IL-12 p40, IL-12 p70, IL-13, IL-15, IL-16, IL-17, MCP-1, M-CSF, MIG, MIP-1 alpha, MIP-1 beta, MIP-1 delta, PDGF-BB, RANTES, TIMP-1, TIMP-2, TNF alpha, TNF beta, sTNFRI, sTNFRIIAR, BDNF, bFGF, BMP-4, BMP-5, BMP-7, b-NGF, EGF, EGFR, EG-VEGF, FGF-4, FGF-7, GDF-15, GDNF, Growth Hormone, HB-EGF, HGF, IGFBP-1, IGFBP-2, IGFBP-3, IGFBP-4, IGFBP-6, IGF-1, Insulin, M-CSF R, NGF R, NT-3, NT-4, Osteoprotegerin, PDGF-AA, PIGF, SCF, SCF R, TGFalpha, TGF beta 1, TGF beta 3, VEGF, VEGFR2, VEGFR3, VEGF-D 6Ckine, Axl, BTC, CCL28, CTACK, CXCL16, ENA-78, Eotaxin-3, GCP-2, GRO, HCC-1, HCC-4, IL-9, IL-17F, IL-18 BPa, IL-28A, IL-29, IL-31, IP-10, I-TAC, LIF, Light, Lymphotactin, MCP-2, MCP-3, MCP-4, MDC, MIF, MIP-3 alpha, MIP-3 beta, MPIF-1, MSPalpha, NAP-2, Osteopontin, PARC, PF4, SDF-1 alpha, TARC, TECK, TSLP 4-1BB, ALCAM, B7-1, BCMA, CD14, CD30, CD40 Ligand, CEACAM-1, DR6, Dtk, Endoglin, ErbB3, E-Selectin, Fas, Flt-3L, GITR, HVEM, ICAM-3, IL-1 R4, IL-1 R1, IL-10 Rbeta, IL-17R, IL-2Rgamma, IL-21R, LIMPII, Lipocalin-2, L-Selectin, LYVE-1, MICA, MICB, NRG1-beta1, PDGF Rbeta, PECAM-1, RAGE, TIM-1, TRAIL R3, Trappin-2, uPAR, VCAM-1, XEDARActivin A, AgRP, Angiogenin, Angiopoietin 1, Angiostatin, Catheprin S, CD40, Cripto-1, DAN, DKK-1, E-Cadherin, EpCAM, Fas Ligand, Fcg RIIB/C, Follistatin, Galectin-7, ICAM-2, IL-13 R1, IL-13R2, IL-17B, IL-2 Ra, IL-2 Rb, IL-23, LAP, NrCAM, PAI-1, PDGF-AB, Resistin, SDF-1 beta, sgp130, ShhN, Siglec-5, ST2, TGF beta 2, Tie-2, TPO, TRAIL R4, TREM-1, VEGF-C, VEGFR1Adiponectin, Adipsin, AFP, ANGPTL4, B2M, BCAM, CA125, CA15-3, CEA, CRP, ErbB2, Follistatin, FSH, GRO alpha, beta HCG, IGF-1 sR, IL-1 sRII, IL-3, IL-18 Rb, IL-21, Leptin, MMP-1, MMP-2, MMP-3, MMP-8, MMP-9, MMP-10, MMP-13, NCAM-1, Nidogen-1, NSE, OSM, Procalcitonin, Prolactin, PSA, Siglec-9, TACE, Thyroglobulin, TIMP-4, TSH2B4, ADAM-9, Angiopoietin 2, APRIL, BMP-2, BMP-9, C5a, Cathepsin L, CD200, CD97, Chemerin, DcR3, FABP2, FAP, FGF-19, Galectin-3, HGF R, IFN-gammalpha/beta ?R2, IGF-2, IGF-2 R, IL-1R6, IL-24, IL-33, Kallikrein 14, Legumain, LOX-1, MBL, Neprilysin, Notch-1, NOV, Osteoactivin, PD-1, PGRP-5, Serpin A4, sFRP-3, Thrombomodulin, TLR2, TRAIL R1, Transferrin, WIF-LACE-2, Albumin, AMICA, Angiopoietin 4, BAFF, CA19-9, CD163, Clusterin, CRTAM, CXCL14, Cystatin C, Decorin, Dkk-3, DLL1, Fetuin A, aFGF, FOLR1, Furin, GASP-1, GASP-2, GCSF R, HAI-2, IL-17B R, IL-27, LAG-3, LDL R, Pepsinogen I, RBP4, SOST, Syndecan-1, TACI, TFPI, TSP-1, TRAIL R2, TRANCE, Troponin I, uPA, VE-Cadherin, WISP-1, and RANK. Additionally, angiogenic factors may be added in some embodiments of the invention. Said angiogenic factors include but are not limited to activin A, adrenomedullin, aFGF, ALK1, ALK5, ANF, angiogenin, angiopoietin-1, angiopoietin-2, angiopoietin-3, angiopoietin-4, bFGF, B61, bFGF inducing activity, cadherins, CAM-RF, cGMP analogs, ChDI, CLAF, claudins, collagen, collagen receptors .alpha..sub.1.beta..sub.1 and .alpha..sub.2.beta..sub.1, connexins, Cox-2, ECDGF (endothelial cell-derived growth factor), ECG, ECI, EDM, EGF, EMAP, endoglin, endothelins, endothelial cell growth inhibitor, endothelial cell-viability maintaining factor, endothelial differentiation shpingolipid G-protein coupled receptor-1 (EDG1), ephrins, Epo, HGF, TGF-beta, PD-ECGF, PDGF, IGF, IL8, growth hormone, fibrin fragment E, FGF-5, fibronectin and fibronectin receptor .alpha.5.beta.1, Factor X, HB-EGF, HBNF, HGF, HUAF, heart derived inhibitor of vascular cell proliferation, IL1, IGF-2 IFN-gamma, integrin receptors, K-FGF, LIF, leiomyoma-derived growth factor, MCP-1, macrophage-derived growth factor, monocyte-derived growth factor, MD-ECI, MECIF, MMP 2, MMP3, MMP9, urokiase plasminogen activator, neuropilin (NRP1, NRP2), neurothelin, nitric oxide donors, nitric oxide synthases (NOSs), notch, occludins, zona occludins, oncostatin M, PDGF, PDGF-B, PDGF receptors, PDGFR-.beta., PD-ECGF, PAI-2, PD-ECGF, PF4, P1GF, PKR1, PKR2, PPAR-gamma, PPAR-gamma ligands, phosphodiesterase, prolactin, prostacyclin, protein S, smooth muscle cell-derived growth factor, smooth muscle cell-derived migration factor, sphingosine-1-phosphate-1 (SIP1), Syk, SLP76, tachykinins, TGF-beta, Tie 1, Tie2, TGF-.beta., and TGF-.beta. receptors, TIMPs, TNF-alphatransferrin, thrombospondin, urokinase, VEGF-A, VEGF-B, VEGF-C, VEGF-D, VEGF-E, VEGF, VEGF.sub.164, VEGI, EG-VEGF.

For the purpose of the invention, bone marrow mononuclear cells/aspirate may be used either freshly isolated, purified, or subsequent to ex vivo culture. A typical bone marrow harvest for collecting starting material for practicing one embodiment of the invention involves a bone marrow harvest with the goal of acquiring approximately 5-700 ml of bone marrow aspirate. Numerous techniques for the aspiration of marrow are described in the art and part of standard medical practice. One particular methodology that may be attractive due to decreased invasiveness is the “mini-bone marrow harvest”. In one specific embodiment bone marrow mononuclear cells are isolated by pheresis or gradient centrifugation. Numerous methods of separating mononuclear cells from bone marrow are known in the art and include density gradients such as Ficoll Histopaque at a density of approximately 1.077 g/ml or Percoll gradient. Separation of cells by density gradients is usually performed by centrifugation at approximately 450 g for approximately 25-60 minutes. Cells may subsequently be washed to remove debris and unwanted materials. Said washing step may be performed in phosphate buffered saline at physiological pH. An alternative method for purification of mononuclear cells involves the use of apheresis apparatus such as the CS3000-Plus blood-cell separator (Baxter, Deerfield, USA), the Haemonetics separator (Braintree, Mass.), or the Fresenius AS 104 and the Fresenius AS TEC 104 (Fresenius, Bad Homburg, Germany) separators. In addition to injection of mononuclear cells, purified bone marrow subpopulations may be used. Additionally, ex vivo expansion and/or selection may also be utilized for augmentation of desired biological properties for use in treatment of ischemic conditions, wherein said cells are administered together with oxytocin. In another embodiment of the invention, the bone marrow aspirate is also directedly injected into the area of alopecia without any processing or filtering.

In the methods of the present invention, autologous bone-marrow is isolated from the subject usually under general anesthesia or local anesthesia by aspiration from the tibia, femur, ilium or sternum with a syringe, preferably containing 1 mL heparin with an 18-gauge needle. Bone-marrow mononuclear cells are isolated using standard techniques with which one of skill is familiar; such techniques may be modified depending upon the species of the subject from which the cells are isolated. The marrow cells are transferred to a sterile tube and mixed with an appropriate amount of medium, e.g., 10 mL culture medium (Iscove's modified Dulbecco medium IMDM with 10% fetal bovine serum, penicillin G [100 U/mL] and streptomycin [100 .mu.g/mL]). The tube is centrifuged to pellet the bone marrow cells, e.g., at 2000 rpm for five minutes and the cell pellet resuspended in medium, e.g., 5 mL culture medium. Low density bone-marrow mononuclear cells are separated from the suspension, e.g., by density gradient centrifugation over Histopaque-1083.™. (Sigma), e.g. as described by Yablonka-Reuveni and Nameroff and hereby incorporated by reference. (Histochemistry (19877) 87:27-38). Briefly, the cell suspension is loaded on 20% to 60% gradient, e.g. Histopaque-1083.™. (Sigma), Ficoll-Hypaque or Percoll (both available from Pharmacia, Uppsala, Sweden) according to manufacturer's instructions and as described by Yablonka-Reuveni and Nameroff. For example, the cells are centrifuged at 400 g for 20 minutes for Ficoll-Hypaque or at 2000 rpm for 10 minutes for Percoll. Following centrifugation, the top two-thirds of total volume are transferred into a tube, as these layers contain most of the low density bone-marrow mononuclear cells. The cells are centrifuged, e.g. at 2000 rpm for 10 minutes to remove the Histopaque. This is repeated and the cell pellet of bone-marrow mononuclear cells is resuspended in culture medium or buffer, e.g., IMDM, saline, phosphate buffered saline, for transplantation. Preferably, fresh bone-marrow mononuclear cell, isolated as described above, are used for transplantation.

This invention provides a method of treating diseased tissue in a subject which comprises: a) isolating autologous bone-marrow mononuclear cells from the subject; and b) transplanting locally into the diseased tissue an effective amount of the autologous bone-marrow mononuclear cells, thereby treating the diseased tissue in the subject. In a preferred embodiment the diseased tissue is ischemic tissue or tissue in need of repair or regeneration. The invention teaches that locally, or systemically in a patient receiving bone marrow mononuclear cell administration results in increasing angiogenesis in diseased tissue in a subject along with decreasing local inflammation.

In some embodiments, the bone-marrow mononuclear cells may also be cultured in any complete medium containing up to 10% serum, e.g., IMDM containing 10% fetal bovine serum and antibiotics, as described above, for up to four weeks before transplantation. The cells may be cultured with growth factors, e.g., vascular endothelial growth factor. The medium is changed about twice a week. The cultured cells are dissociated from the culture dishes with 0.05% trypsin (Gibco BRL, Grand Island, N.Y.), neutralized with culture medium and collected by centrifugation, for example, at 2000 rpm for five minutes at room temperature. The cells are resuspended in IMDM at a concentration of .apprxeq.1.times.10.sup.5 cells to about 1.times.10.sup.10 cells, preferably about 1.times.10.sup.7 cells to about 1.times.10.sup.8 cells in 50 .mu.L for transplantation.

In some embodiments bone marrow cells, alone or in combination with various adjuvants described herein are utilized to enhance efficacy of hair transplantation. Hair techniques have also been developed in an effort to improve the issues discussed above, and include methods for in vitro reproduction of hair or hair cloning. Such methods exploit the characteristics of the hair follicle. The essential growth structures of hair are the hair follicles, which contain hair follicular stem cells responsible for the growth of a new hair. The hair follicles also produce hair follicle cells or keratinocytes. During their journey to the surface of the skin, the cytoplasm of the hair follicle cells undergoes a large number of complex processes, which ultimately lead to the production of the tough and elastic material known as hair. The growth cycle of hair can be subdivided into three phases including the anagen phase (‘growth phase’), the catagen phase (‘transitional phase’), and the telogen phase (‘death phase’). The hair follicle plays a unique role in the cyclic nature of hair formation and hair growth, since it is the only part of the body that has the ability to completely regenerate (i.e. produce a new hair) after its removal from the body. This knowledge has been tested in vitro where it has been shown that hair follicle cells from plucked human hair can be cultured outside the body. It is also known that it is possible to use such cultured cells to form a differentiated epidermis or a fully developed epidermis, both in vitro and in vivo. AA hair transplant method exploiting this concept has been disclosed in European Patent Application 0236 014, in which epidermal follicle cells of the desired hair type are removed from the scalp skin of a donor subject. The epidermal follicle cells are then cultured in a culture medium, which preferably contains growth factors. In a subsequent step, a opening is made in the epidermis of the patients scalp and, via said opening, the cultured epidermal follicle cells are introduced/injected into the dermis next to the epidermis. Although an improvement over the more crude hair transplant method discussed above, the disadvantage of this method is that it is a very invasive procedure and that epidermal follicle cells cannot easily be placed in a targeted manner (i.e. to achieve a specific growth direction) on the scalp or other facial areas. In addition to that, the probability that the injected epidermal follicle cells will regenerate into the recipient region is rather low and as a result, large amount of epidermal follicle cells are required to increase the likelihood of survival. This represents an important limiting factor since such cells are not easily obtained and are difficult to culture in vitro. Another hair transplantation method relying on the concept of hair multiplication has been described in European patent application 0 971 679. In this method, the donor hair in the anagen phase is removed from a donor area in such a way that the growth of a new hair (to replace the plucked hair), is enabled in the donor area. This method can be used to produce a new hair from the hair follicle stern cells obtained from the harvested hair. However, for this technique to succeed, the hair follicle stem cells must be cultured for long periods (ranging from 1 hour to 40 days, in a serum-free keratinocyte culture medium) before the donor hair can be implanted in the recipient area and produce a new hair. Therefore, a main disadvantage of this method is the long time needed for culturing the hair follicle stem cells, causing inconveniency to the recipient subject, who must return to the clinic frequently in order to finalize the procedure. The invention aims to overcome this limitation.

The utilization of platelet rich plasma in the current invention be it from autologous or allogeneic sources when injected into the effected areas of alopecia enable increase nutrients and trophic factors into the treatment area. In one embodiment, the injections are performed via micro-needling. The platelet rich plasma when leuko-rich, increases the remodeling and healing abilities in treated area due to the high concentration of monocytes and granulocytes. In some cases where a high inflammatory state exists in the patient a leuko-depleted platelet rich plasma is used. In both cases the pre/cotreatment with the bone marrow cells/aspirate increases survival of current hair follicles and contributes to generation of new follicles. This occurs via angiogenesis and increased T regulatory cells which enable survival and decreased local inflammation. When these are administered pre and post treatment with laser therapy is also utilized, where follicles' are activated into growth phase, there is an accelerated rate of hair growth and return.

1. A method of preventing hair loss and/or stimulation of new hair growth comprising the steps of: a) identifying a patient in need of treatment; b) providing a bone marrow mononuclear cell/aspirate population into the area of affliction; c) administering platelet rich plasma into the area of affliction; and d) providing adjuvant treatments.

2. The method of claim 1, wherein administration of low-level laser irradiation is performed to augment production of local growth factors pre and/or post cell administration.

3. The method of claim 1, wherein said hair loss is caused by conditions selected from the group consisting of: a) Involutional alopecia; b) Androgenic alopecia; c) Alopecia areata; d) Alopecia universalis; e) Telogen effluvium; and f) Scarring alopecias.

4. The method of claim 1, wherein said hair follicle adjuvant treatment is flutamide.

5. The method of claim 1, wherein said hair follicle adjuvant treatment is finasteride.

6. The method of claim 1, wherein said hair follicle adjuvant treatment is a thiane-1-oxide.

7. The method of claim 1, wherein said hair loss is associated with a reduction in T regulatory cell number.

8. The method of claim 7, wherein said T regulatory cells express Fas ligand.

9. The method of claim 1, wherein said platelet rich plasma is generated from peripheral blood.

10. The method of claim 1, wherein said platelet rich plasma is generated from umbilical cord blood.

11. The method of claim 1, wherein said platelet rich plasma is generated from mobilized peripheral blood.

12. The method of claim 1, wherein said platelet rich plasma contains >10 pg/ml of interleukin 10.

13. The method of claim 1, wherein the adjuvant treatment is an immune suppressive agent that is administered prior to, concurrent with or subsequent to stem cell administration.

14. The method of claim 13, wherein said immune suppressive agent is tacrolimus.

15. The method of claim 13, wherein said immune suppressive agent is rituximab.

16. The method of claim 1, wherein the patient has undergone or is scheduled to undergo a hair transplantation.

17. The method of claim 1, wherein the said platelet rich plasma has a hematocrit of less than 5%.

18. The method of claim 1, wherein the said platelet rich plasma is transformed into lyophilized platelet lysate.

19. The method of claim 18, where the lyophilized platelet lysate is autologous.

20. The method of claim 18, where the lyophilized platelet lysate is allogeneic.
